(19) United States
(12) Patent Application Publication
(10) Pub. No.: US 2010/0233261 A1
(43) Pub. Date: Sep. 16, 2010

(54) Pharmaceutical Composition Comprising Amlodipine and Losartan

(75) Inventors: Jong Soo Woo, Suwon-si (KR); Hong Gi Yi, Suwon-si (KR); Moon Hyuk Chi, Suwon-si (KR); Kyeong Soo Kim, Suwon-si (KR)

Correspondence Address: SUGHRUE MION, PLLC, 2100 PENNSYLVANIA AVENUE, N.W., SUITE 800, WASHINGTON, DC, 20037, US

(73) Assignee: HANMI PHARM. CO., LTD., Hwaseong-gun (KR)

(21) Appl. No.: 12/303,034

(22) PCT Filed: Dec. 7, 2007
(86) PCT No.: PCT/KR07/06352

§ 371 Date: Dec. 1, 2008

(30) Foreign Application Priority Data
Dec. 8, 2006 (KR) 10-2006-0124552

(51) Int. Cl. A61K 9/30; A61K 31/455; A61K 9/00; A61K 9/20; A61P 9/00; A61P 9/10; A61P 9/12

(52) U.S. Cl. 424/475; 514/356; 424/400; 424/464

(57) ABSTRACT

Disclosed herein is a pharmaceutical composition comprising a part containing amlodipine or a pharmaceutically acceptable salt thereof and another separate part containing losartan or a pharmaceutically acceptable salt thereof, which exhibits improved preventive and therapeutic effects against cardiovascular disorders.

### FIELD OF THE INVENTION

The present invention relates to a composition for preventing or treating cardiovascular disorders comprising amlodipine or a pharmaceutically acceptable salt thereof and losartan or a pharmaceutically acceptable salt thereof, and a method for preparing the same.

### BACKGROUND OF THE INVENTION

Hypertension can be classified into essential or secondary hypertension, and most (approximately 90-95%) of the hypertension belongs to the essential hypertension class. While secondary hypertension is generally treatable by correcting the known causes, essential hypertension, the exact cause of which is not yet elucidated, is generally treated by relaxation therapy, dietary therapy and exercise therapy which are optionally combined with medication. Notable antihypertensive drugs include diuretics, sympatholytic agents and vasodilators. Vasodilators are most widely prescribed antihypertensive drugs, and they are divided into several groups according to their pharmacological action which include ACE (angiotensin converting enzyme) inhibitors, angiotensin II receptor antagonists and calcium channel blockers.

In the treatment of hypertension, it is more important to maintain the blood pressure within a normal range on a consistent basis than to simply lower the blood pressure level itself, for reducing the risks of complications such as coronary heart diseases and cardiovascular diseases, e.g., stroke, heart failure and myocardial infarction. Accordingly, antihypertensive agents should be effective for long-term treatment of hypertension. Further, advanced therapy using a combination of two or more drugs having different pharmacological actions makes it possible to improve preventive or therapeutic effects, while lowering side effects arising from the long term administration of a single drug.

The present inventors have therefore endeavored to develop an improved composition for preventing or treating cardiovascular disorders that is free from the above problems, and have found that a pharmaceutical composition which comprises amlodipine or a pharmaceutically acceptable salt thereof and losartan or a pharmaceutically acceptable salt thereof having different pharmacological activities.

### SUMMARY OF THE INVENTION

Accordingly, it is an object of the present invention to provide a composition for the prevention and treatment of cardiovascular disorders, which comprises amlodipine or a pharmaceutically acceptable salt thereof and losartan or a pharmaceutically acceptable salt thereof having different pharmacological activities.

It is another object of the present invention to provide a method for preparing said composition.

In accordance with one aspect of the present invention, there is provided a composition for preventing and treating cardiovascular disorders comprising amlodipine or a pharmaceutically acceptable salt thereof and losartan or a pharmaceutically acceptable salt thereof.

### BRIEF DESCRIPTION OF THE DRAWINGS

The above and other objects and features of the present invention will become apparent from the following description of the invention, when taken in conjunction with the accompanying drawings which respectively show:

FIG. 1: a graph showing the amlodipine dissolution rate, obtained in Test Example 1;

FIG. 2: a graph showing the losartan dissolution rate, obtained in Test Example 2;

FIG. 3: a graph showing the amlodipine dissolution rate, obtained in Test Example 3;

FIG. 4: a graph showing the amlodipine dissolution rate, obtained in Test Example 4; and

FIG. 5: a graph showing the amlodipine dissolution rate, obtained in Test Example 5.

### DETAILED DESCRIPTION OF THE INVENTION

Amlodipine is the generic name for 3-ethyl-5-methyl-2-(2-aminoethoxy-methyl)-4-(2-chlorophenyl)-6-methyl-1,4-dihydro-3,5-pyridine dicarboxylate, and EP Patent Publication No. 89167 discloses various forms of pharmaceutically acceptable salts of amlodipine. The pharmaceutically acceptable salts of amlodipine used in the present invention may be formed using acids which form non-toxic, pharmaceutically acceptable acid addition salts, which include but are not limited to hydrochloride, hydrobromide, sulphate, phosphate, acetate, maleate, fumarate, lactate, tartrate, citrate, gluconate, besylate and camsylate salts. Among the acid addition salts, amlodipine besylate is currently marketed as Novasc (trade mark), and Korean Patent No. 452491 has disclosed the camsylate salt of amlodipine having improved physical properties, i.e., higher solubility and stability as compared with amlodipine besylate. Accordingly, the most preferred pharmaceutically acceptable salt of amlodipine that can be used in the present invention is amlodipine camsylate. Amlodipine is a long-acting calcium channel blocker which is useful in treating cardiovascular disorders such as agina, hypertension and congestive heart failure. However, it has been reported that prolonged anti-hypertensive therapy with amlodipine often causes side effects such as dose-limiting peripheral edema, especially ankle edema. The amlodipine-induced ankle edema, for example, is believed to be due the the preferential dilation of the precapillary arterioles in the leg and the resultant efflux of fluid into the interstitial space. A daily proposed dose of amlodipine or a pharmaceutically acceptable salt thereof is 0.5˜20 mg, preferably 1˜10 mg, more preferably 5˜10 mg.

Losartan is the generic name for 2-butyl-4-chloro-1-[[2′-(1H-tetrazol-5-yl)[1,1′-biphenyl]-4-yl]methyl]-1H-imidazol-5-methanol, which has been disclosed in U.S. Pat. Nos. 5,608,075; 5,138,069; and 5,153,197. Losartan potassium is currently available as Cozaar (trade mark). Accordingly, the preferred pharmaceutically acceptable salt of losartan that can be used in the present invention is losartan potassium. Losartan blocks the interaction of angiotensin II and its receptor, and is mainly used for treating hypertension, heart failure, ischemic peripheral circulatory disorder, myocardial ischemia (angina pectoris), diabetic neuropathy and glaucoma, and also for preventing the progression of post-myocardial infarction heart failure. Although losartan is reported to cause low incidence of cough or edema, it sometimes induces side effects such as dizziness or orthostatic hypotension. A daily proposed dose of losartan or a pharmaceutically acceptable salt thereof is 0.1˜500 mg, preferably 1˜200 mg, more preferably 25˜200 mg.

The inventive composition comprising amlodipine or a pharmaceutically acceptable salt thereof and losartan or a pharmaceutically acceptable salt thereof can achieve improved preventive or therapeutic effects for cardiovascular disorders, such as angina pectoris, hypertension, artery vasospasm, deep vein, cardiac hypertrophy, cerebral infarct, congestive heart failure and myocardial infarction, as compared with conventional single formulations, while minimizing adverse effects of the two drugs.

However, the combined formulation of amlodipine and losartan of the present invention cannot be prepared simply by mixing the two drugs for the following problems.

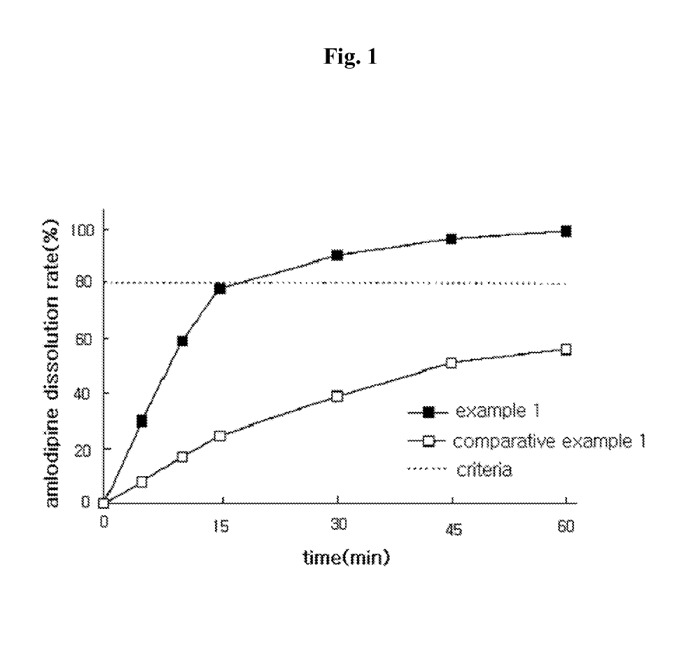

The first problem is gelation of losartan. Losartan dissolves readily in water of at high pH, e.g., pH 4.0 or pH 6.8, but it is very slowly released in an aqueous medicament at low pH (e.g., pH 2.0 or pH 1.2) because of its gelation. Accordingly, the dissolution rate and bioavailability of the combined formulation comprising losartan are expected to be unsatisfactory because the formulation is first exposed to the acidic gastronic fluid having low pH value when orally administered. Further, amlodipine may be locked in the inside of the formulation due to the gelation of losartan. For example, as can be seen in FIG. 1, which is the results of Test Example 1, a tablet prepared by simply mixing the two drugs fails to meet the dissolution criteria of amlodipine, i.e., 80% at 30 mins. Therefore, an acceptable combined formulation must be free from the problem of losartan gelation even under a low pH condition.

The second problem is that the drug stability of a simple combined formulation mixing the two drugs decreases rapidly due to the adverse influence of losartan on the drug stability of amlodipine.

Accordingly, the present invention also includes within its scope a combined formulation of amlodipine and losartan in which the contact between the two drugs is minimized by physically separating amlodipine or a pharmaceutically acceptable salt thereof from losartan or a pharmaceutically acceptable salt thereof, thereby improving the dissolution rates and stabilities of amlodipine and losartan.

In accordance with one embodiment of the present invention, the amlodipine-losartan combined formulation may be prepared by using a separate granule or form of amlodipine in order to physically separate amlodipine from losartan in the formulation. That is, the inventive combined formulation may be prepared by a method comprising the steps of 1) granulating amlodipine or a pharmaceutically acceptable salt thereof to obtain separated granules; and 2) mixing the separated granule part with a mixture comprising losartan or a pharmaceutically acceptable salt thereof. The combined formulations of Examples 1 to 4 prepared by this method exhibit an enhanced dissolution rate of amlodipine while maintaining a satisfactory dissolution rate of losartan (see FIGS. 1 to 3), and also exhibit high drug stability of amlodipine (see Table 1), as compared with the combined formulation of Comparative Example 1, which is a tablet obtained using a simple mixture of amlodipine or a pharmaceutically acceptable salt thereof and losartan or a pharmaceutically acceptable salt thereof.

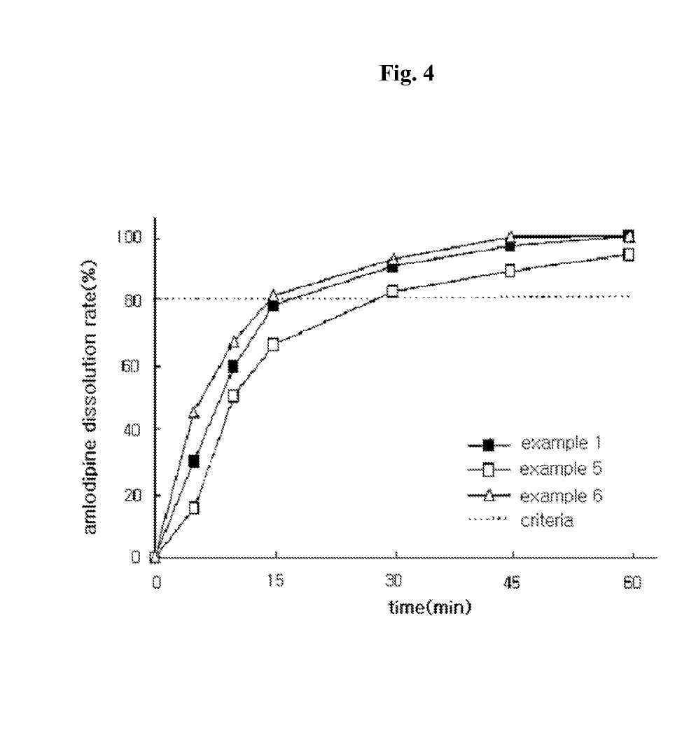

In accordance with another embodiment of the present invention, the inventive combined formulation may take the form of a two-layer tablet of amlodipine or a pharmaceutically acceptable salt thereof and losartan or a pharmaceutically acceptable salt thereof. As described in Example 8, the two-layer tablet may be prepared by formulating separated granules of amlodipine into a tablet with a two-layer tablet press machine, mixing the tablet with a mixture comprising losartan, and formulating the resulting mixture into a two-layer tablet. The two-layer tablet thus obtained also exhibits an improved amlodipine dissolution rate similarly to that observed for the formulation of Example 1, as shown in FIG. 4.

In accordance with still another embodiment of the present invention, the inventive combined formulation or two-layer tablet prepared by using amlodipine granules may further comprise a coating layer between the amlodipine and losartan components, which may be prepared by a method comprising the steps of spraying a coating material on separated granules of amlodipine, drying the coated granules, and mixing the coated granule with losartan.

In accordance with yet another embodiment of the present invention, the inventive formulation in which amlodipine and losartan are separated from each other, may take the form of a losartan tablet coated with amlodipine. Such a tablet may be prepared by a method comprising the steps of dissolving or dispersing amlodipine in a coating solution, and coating a tablet of losartan with the resulting dispersant. This tablet may further comprise a separating layer between the losartan tablet and the amlodipine coating layer. The coating solution used in the procedure of dispersing amlodipine may be a solution of one of the known coating materials, which may include but are not limited to methyl cellulose (MC), ethyl cellulose (EC), hydroxyethyl cellulose (HEC), hydroxypropylmethyl cellulose (HPMC) and polyvinylpyrrolidone (PVP).

Accordingly, in a further embodiment, the present invention also includes within its scope a composition for preventing or treating cardiovascular disorders comprising amlodipine or a pharmaceutically acceptable salt thereof and losartan or a pharmaceutically acceptable salt thereof, the part comprising amlodipine or a pharmaceutically acceptable salt thereof being separated from the part containing losartan or a pharmaceutically acceptable salt thereof.

In yet another embodiment, the present invention also includes within its scope a composition for preventing or treating cardiovascular disorders comprising amlodipine or a pharmaceutically acceptable salt thereof and losartan or a pharmaceutically acceptable salt thereof, the composition being prepared by separately granulating amlodipine or a pharmaceutically acceptable salt thereof to obtain an amlodipine granule part, and mixing the amlodipine granule part with a mixture comprising losartan or a pharmaceutically acceptable salt thereof. The composition of the present invention may further comprise a coating layer coated on the amlodipine granule part.

In yet another embodiment, the present invention also includes within its scope a composition for preventing or treating cardiovascular disorders comprising amlodipine or a pharmaceutically acceptable salt thereof and losartan or a pharmaceutically acceptable salt thereof, the composition being in the form of a two-layer tablet in which amlodipine or a pharmaceutically acceptable salt thereof and losartan or a pharmaceutically acceptable salt thereof form separate distinct layers. Further, the inventive two-layer tablet may further comprise a coating layer formed on the amlodipine layer.

In yet another embodiment, the present invention also includes within its scope a composition for preventing or treating cardiovascular disorders comprising amlodipine or a pharmaceutically acceptable salt thereof and losartan or a pharmaceutically acceptable salt thereof, which is in the form of a tablet of losartan or the pharmaceutically acceptable salt thereof coated with amlodipine or a pharmaceutically acceptable salt thereof. Further, the composition of the present invention may further comprise a separating layer disposed between the losartan tablet and the amlodipine coating.

In the inventive composition for preventing or treating cardiovascular disorders, the amlodipine granules and the mixture comprising losartan may respectively comprise pharmaceutically acceptable carriers or excipients. The pharmaceutically acceptable carriers or excipients may include microcrystalline cellulose, lactose, sodium citrate, calcium phosphate, glycine, starch, disintegrants (e.g., sodium starch glycolate, croscarmellose sodium and composite silicate) and granulating binders (e.g., polyvinylpyrrolidone, hydroxypropylmethylcellulose (HPMC), hydroxypropylcellulose (HPC), sucrose, gelatine and acacia gum). Further, the inventive composition may further comprise lubricants such as magnesium stearate, stearic acid, glyceryl behenate and talc.

In a preferred embodiment of the present invention, the amlodipine granule part may comprise amlodipine or a pharmaceutically acceptable salt thereof and excipients in amounts corresponding to a weight ratio in the range of 1:10 to 1:60. When the weight ratio is less than 1:10, the dissolution rate of the composition becomes poor (see Example 5-7 and FIG. 4), and when more than 1:60, the composition becomes too bulky for easy administration.

Further, in the composition of the present invention, amlodipine or a pharmaceutically acceptable salt thereof and losartan or a pharmaceutically acceptable salt thereof may be used in amounts corresponding to a weight ratio in the range of 1:2.5 to 1:20, preferably 1:5 to 1:10.

The composition of the present invention may be administered in the form of a tablet, a capsule or multi-particles through various routes of oral administration including oral cavity, mouth and hypoglossus. However, it should be understood that the administration route of the inventive composition should be determined by the doctor in charge based on the patient's symptoms and requirements.

In accordance with a further aspect of the present invention, there is provided a method for preparing the inventive pharmaceutical composition comprising amlodipine or a pharmaceutically acceptable salt thereof and losartan or a pharmaceutically acceptable salt thereof, which comprises the steps of:

a) wet-granulating and drying a mixture of amlodipine or a pharmaceutically acceptable salt thereof and pharmaceutically acceptable excipients to obtain an amlodipine granule part; and

b) mixing the amlodipine granule part obtained in step a) with a mixture part comprising losartan or a pharmaceutically acceptable salt thereof and pharmaceutically acceptable excipients.

The following Examples are intended to further illustrate the present invention without limiting its scope.

### Example 1

### Preparation of Combined Tablet I

Amlodipine granule part

| | | |
|---|---|---|
| amlodipine camsylate | 7.84 | mg (amlodipine 5 mg) |
| microcrystalline cellulose | 70.0 | mg |
| calcium dihydrogenous phosphate | 60.0 | mg |
| sodium starch glycolate | 12.0 | mg |
| povidone | 3.0 | mg |
| purified water | 70.0 | mg |

Losartan mixture part

| | | |
|---|---|---|
| losartan potassium | 50.0 | mg |
| microcrystalline cellulose | 80.0 | mg |
| sodium starch glycolate | 12.0 | mg |
| magnesium stearate | 2.0 | mg |

The ingredients of the amlodipine granule part were wet-granulated using 70.0 mg/tablet of purified water, passed through a 20 mesh, and dried to obtain the granule part having the specified amounts of the ingredient. The dried amlodipine granule part was mixed with the ingredients of the losartan mixture part according to the corresponding amounts, and the resulting mixture was formulated into a combined tablet having 5 mg of amlodipine and 50 mg of losartan.

### Example 2

### Preparation of Combined Tablet II

Amlodipine granule part

| | | |
|---|---|---|
| amlodipine camsylate | 7.84 | mg (amlodipine 5 mg) |
| mannitol | 70.0 | mg |
| calcium dihydrogenous phosphate | 60.0 | mg |
| sodium starch glycolate | 12.0 | mg |
| povidone | 3.0 | mg |
| purified water | 70.0 | mg |

Losartan mixture part

| | | |
|---|---|---|
| losartan potassium | 50.0 | mg |
| mannitol | 80.0 | mg |
| sodium starch glycolate | 12.0 | mg |
| magnesium stearate | 2.0 | mg |

A combined tablet containing 5 mg of amlodipine and 50 mg of losartan was prepared by repeating the procedure of Example 1 except for using mannitol instead of microcrystalline cellulose.

### Example 3

### Preparation of Combined Tablet III

Amlodipine granule part

| | | |
|---|---|---|
| amlodipine camsylate | 15.68 | mg |
| microcrystalline cellulose | 140.0 | mg |
| calcium dihydrogenous phosphate | 120.0 | mg |
| sodium starch glycolate | 24.0 | mg |
| povidone | 6.0 | mg |
| purified water | 140.0 | mg |

Losartan mixture part

| | | |
|---|---|---|
| losartan potassium | 50.0 | mg |
| microcrystalline cellulose | 80.0 | mg |
| sodium starch glycolate | 12.0 | mg |
| magnesium stearate | 2.0 | mg |

A combined tablet containing 10 mg of amlodipine and 50 mg of losartan was prepared by repeating the procedure of Example 1 except for using the ingredients described in amlodipine granule part twice the corresponding amounts, respectively.

### Example 4

### Preparation of Combined Tablet IV

Amlodipine granule part

| | | |
|---|---|---|
| amlodipine camsylate | 15.68 | mg |
| microcrystalline cellulose | 140.0 | mg |
| calcium dihydrogenous phosphate | 120.0 | mg |
| sodium starch glycolate | 24.0 | mg |
| povidone | 6.0 | mg |
| purified water | 140.0 | mg |

Losartan mixture part

| | | |
|---|---|---|
| losartan potassium | 100.0 | mg |
| microcrystalline cellulose | 160.0 | mg |
| sodium starch glycolate | 24.0 | mg |
| magnesium stearate | 4.0 | mg |

A combined tablet containing 10 mg of amlodipine and 100 mg of losartan was prepared by repeating the procedure of Example 3 except for using the ingredients described in losartan mixture part twice the corresponding amounts, respectively.

### Example 5

### Preparation of Combined Tablet V

Amlodipine granule part

| | | |
|---|---|---|
| amlodipine camsylate | 7.84 | mg (amlodipine 5 mg) |
| microcrystalline cellulose | 35.0 | mg |
| calcium dihydrogenous phosphate | 30.0 | mg |
| sodium starch glycolate | 6.0 | mg |
| povidone | 1.5 | mg |
| purified water | 35.0 | mg |

Losartan mixture part

| | | |
|---|---|---|
| losartan potassium | 50.0 | mg |
| microcrystalline cellulose | 80.0 | mg |
| sodium starch glycolate | 12.0 | mg |
| magnesium stearate | 2.0 | mg |

A combined tablet was prepared by repeating the procedure of Example 1 except for using the rest excipients in an amount of 10 parts by weight based on 1 part by weight of amlodipine camsylate in the procedure of preparing the amlodipine granule part.

### Example 6

### Preparation of Combined Tablet VI

Amlodipine granule part

| | | |
|---|---|---|
| amlodipine camsylate | 7.84 | mg (amlodipine 5 mg) |
| microcrystalline cellulose | 231.0 | mg |
| calcium dihydrogenous phosphate | 198.0 | mg |
| sodium starch glycolate | 40.0 | mg |
| povidone | 10.0 | mg |
| purified water | 462.0 | mg |

Losartan mixture part

| | | |
|---|---|---|
| losartan potassium | 50.0 | mg |
| microcrystalline cellulose | 80.0 | mg |
| sodium starch glycolate | 12.0 | mg |
| magnesium stearate | 2.0 | mg |

A combined tablet was prepared by repeating the procedure of Example 1 except for using the rest excipients in an amount of 60 parts by weight based on 1 part by weight of amlodipine camsylate in the procedure of preparing the amlodipine granule part.

### Example 7

### Preparation of Combined Tablet VII

Amlodipine granule part

| | | |
|---|---|---|
| amlodipine camsylate | 15.68 | mg (amlodipine 10 mg) |
| microcrystalline cellulose | 462.0 | mg |
| calcium dihydrogenous phosphate | 396.0 | mg |
| sodium starch glycolate | 80.0 | mg |
| povidone | 20.0 | mg |
| purified water | 924.0 | mg |

Losartan mixture part

| | | |
|---|---|---|
| losartan potassium | 50.0 | mg |
| microcrystalline cellulose | 80.0 | mg |
| sodium starch glycolate | 12.0 | mg |
| magnesium stearate | 2.0 | mg |

A combined tablet was prepared by repeating the procedure of Example 3 except for using the rest excipients in an amount of 60 parts by weight based on 1 part by weight of amlodipine camsylate in the procedure of preparing the amlodipine granule part.

### Example 8

### Preparation of Two-Layer Tablet

Amlodipine tablet part

| | | |
|---|---|---|
| amlodipine camsylate | 7.84 | mg (amlodipine 5 mg) |
| microcrystalline cellulose | 70.0 | mg |
| calcium dihydrogenous phosphate | 60.0 | mg |
| sodium starch glycolate | 12.0 | mg |
| povidone | 3.0 | mg |
| purified water | 70.0 | mg |

Losartan mixture part

| | | |
|---|---|---|
| losartan potassium | 50.0 | mg |
| microcrystalline cellulose | 80.0 | mg |
| sodium starch glycolate | 12.0 | mg |
| magnesium stearate | 2.0 | mg |

The ingredients described in amlodipine tablet part were wet-granulated using 70.0 mg/tablet of purified water, passed through a 20 mesh, and dried, according to the corresponding amounts. The dried amlodipine granule part was formulated into a tablet by using a two-layer tablet press machine (MRC-45, Sejong Pharmatech), the ingredients described in Losartan mixture part were added thereto according to the corresponding amounts, and the resulting mixture was formulated into a two-layer tablet containing 5 mg of amlodipine and 50 mg of losartan.

### Comparative Example 1

### Preparation of Direct-Compression Tablet of Non-Separated Amlodipine and Losartan

| | | |
|---|---|---|
| amlodipine camsylate | 7.84 | mg (amlodipine 5 mg) |
| microcrystalline cellulose | 150.0 | mg |
| calcium dihydrogenous phosphate | 60.0 | mg |
| sodium starch glycolate | 24.0 | mg |
| povidone | 3.0 | mg |
| losartan potassium | 50.0 | mg |
| magnesium stearate | 2.0 | mg |

All ingredients described in Example 1 were mixed together according to the corresponding amounts, and the mixture was formulated into a direct-compression tablet containing 5 mg of amlodipine and 50 mg of losartan.

### Test Example 1

### Dissolution Test of Amlodipine

The combined tablet prepared in Example 1 and the tablet of a non-separated mixture prepared in Comparative Example 1 were each subjected to a drug dissolution test under the following conditions.

—Test Conditions—
Effluent: 500 ml of 0.01 N HCl (pH 2.0)
Dissolution-test system: USP paddle method, 75 rpm
Temperature: 37° C.

—Analytical Conditions—
Column: stainless steel column (inner diameter: 4.6 mm, length: 25 cm) filled with octadecylsilanized silica gel for 5 μm liquid chromatography
Mobile phase: a mixture of methonol and 0.03M potassium dihydrogenous phosphate (600:400, v/v)
Detector: ultraviolet spectrophotometer (237 nm)
Flow rate: 1.5 ml/min
Injection volume: 20 μl

—Criteria of Dissolution Rate—
more than 80% at 30 mins.

—Results—

As shown in FIG. 1, the combined tablet of amlodipine-losartan prepared in Example 1 exhibited an amlodipine dissolution rate two times higher as compared with that of the direct-compression tablet of a non-separated mixture prepared in Comparative Example 1. Further, the dissolution rate of the tablet prepared in Comparative Example 1 did not satisfy the required criteria, while that of the tablet of Example 1 met the criteria.

### Test Example 2

### Dissolution Test of Losartan

The combined tablet prepared in Example 1 and the tablet of a non-separated mixture prepared in Comparative Example 1 were each subjected to a drug dissolution test under the following conditions.

—Test Conditions—
Effluent: purified water
Dissolution-test system: USP paddle method, 50 rpm
Temperature: 37° C.

—Analytical Conditions—
Column: stainless steel column (inner diameter: 4.6 mm, length: 25 cm) filled with octadecylsilanized silica gel for 5 μm liquid chromatography
Mobile phase:
   mobile phase A—phosphate buffer:acetonitrile (850:150, v/v)
   mobile phase B—acetonitrile
   concentration gradient system

| Time (min) | Mobile phase A % | Mobile phase B % |
|---|---|---|
| 0 | 80 | 20 |
| 10 | 40 | 60 |
| 11 | 80 | 20 |
| 15 | 80 | 20 |

Detector: ultraviolet spectrophotometer (250 nm)
Flow rate: 1.5 ml/min
Injection volume: 10 μl

—Criteria of Dissolution Rate—
more than 85% at 45 mins.

—Results—

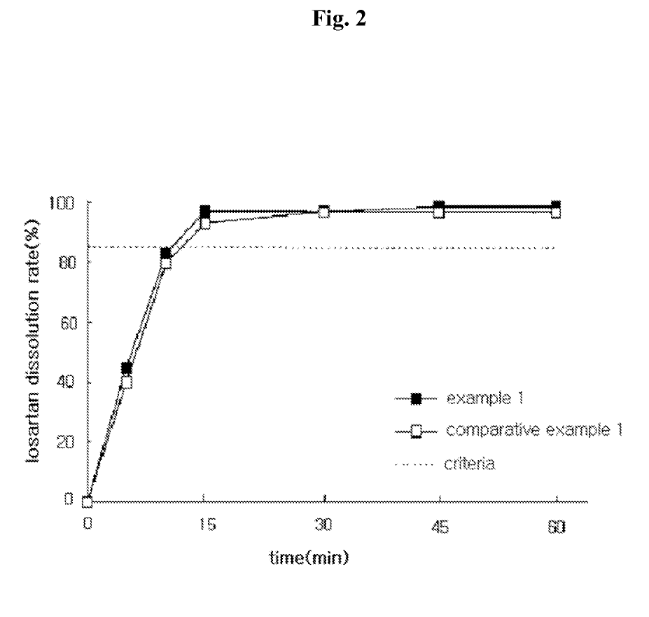

As shown in FIG. 2, the combined tablet of amlodipine-losartan prepared in Example 1 and the tablet of a non-separated mixture prepared in Comparative Example 1 did not show any significant difference in the dissolution rates of losartan, and all the two tablets met the criteria. This suggests that the separate granulation of amlodipine does not affect the dissolution rate of losartan.

### Test Example 3

### Dissolution Test of Amlodipine for the Formulations of Examples 1 to 4

The combined tablets of Examples 1 to 4, which were prepared by using a separated granule, respectively, were each subjected to drug dissolution test under the same test and analytical conditions of Test Example 1.

—Results—

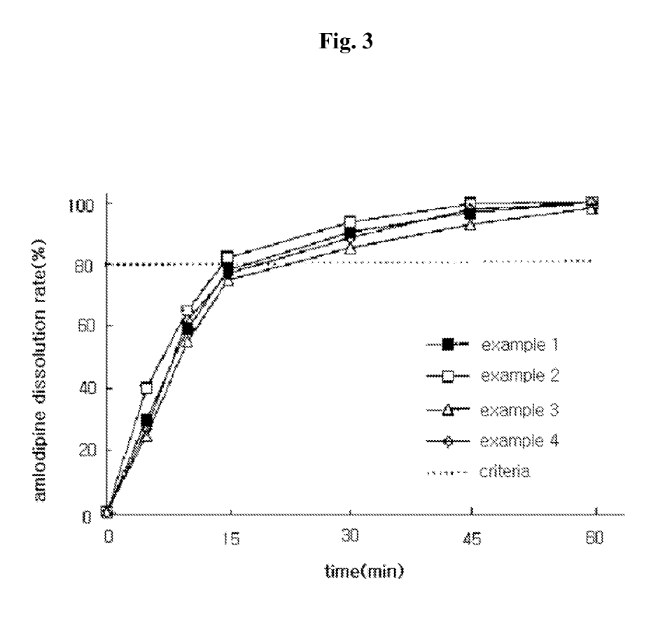

As can be seen in FIG. 3, the combined tablets prepared in Examples exhibited similar amlodipine dissolution rates regardless of the difference in the content or kind of ingredients.

### Test Example 4

### Dissolution Test of Amlodipine for the Formulations of Examples 1, 5 and 6

The combined tablets prepared in Examples 1, 5 and 6 were each subjected to drug dissolution test under the same test and analytical conditions of Test Example 1.

—Results—

As shown in FIG. 4, the tablet obtained in Example 6, which was prepared by using the rest excipients in an amount of 60 parts by weight based on 1 part by weight of amlodipine camsylate, exhibited a similar amlidipine dissolution rate to that of the formulation obtained in Example 1, while the tablet of Example 5, which was prepared by using the rest excipients in an amount of 10 parts by weight based on 1 part by weight of amlodipine camsylate, showed a slightly lower dissolution rate than that of the formulation of Example 1. The dissolution rate at 30 min of the formulation obtained in Example 5 is 80%, which met the criteria, and accordingly, it can be expected that the dissolution rate of the inventive formulation would become unsatisfactory when the amount of the rest excipients is less than 10 parts by weight.

### Test Example 5

### Dissolution Test of Amlodipine for the Formulations of Examples 1 and 8

The combined tablet obtained in Example 1 and the two-layer tablet obtained in Example 8 were each subjected to drug dissolution test under the same test and analytical conditions of Test Example 1.

—Results—

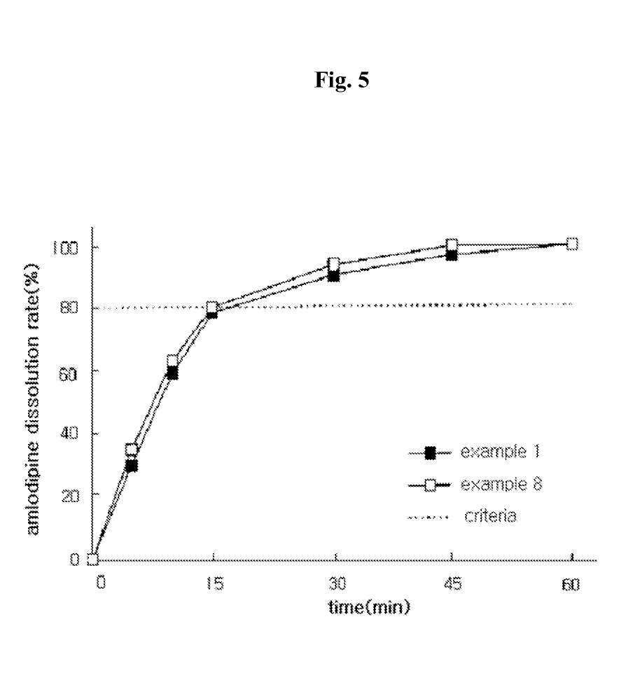

As shown in FIG. 5, the combined tablet obtained in Example 1 and the two-layer tablet obtained in Example 8 exhibited similar dissolution rates regardless of the difference in the forms of the formulation. Accordingly, this suggest that the inventive formulation prepared by using a separated granule of amlodipine or a pharmaceutically acceptable salt thereof would exhibit a satisfactory dissolution rate irrespective of the formulation form.

### Test Example 6

### Stability Test of Amlodipine

Stability test was performed for the combined tablet obtained in Example 1, which was prepared by using a separated granule, and the tablet of a non-separated mixture obtained in Comparative Example 1 under the following conditions.

Incubation conditions: HDPE bottle at 40° C./75% relative humidity

Incubation time: 0, 1, 2, 4 and 6 months

Subject of test: amlodipine

Analytical conditions: the analytical conditions of Example 1

The results are shown in Table 1.

TABLE 1

| Formulation | 0 | 1 month | 2 months | 4 months | 6 months |
|---|---|---|---|---|---|
| Example 1 | 100.3% | 100.2% | 99.6% | 98.9% | 99.1% |
| Comparative | 100.2% | 97.8% | 94.9% | 90.3% | 85.7% |
| Example 1 | | | | | |

As shown in Table 1, the combined tablet obtained in Example 1, which was prepared by using a separated granule, exhibited a high drug stability as compared with the tablet of a non-separated mixture obtained in Comparative Example 1.

As described above, the inventive composition comprising amlodipine or a pharmaceutically acceptable salt thereof and losartan or a pharmaceutically acceptable salt thereof can achieve improved therapeutic effects for cardiovascular disorders. Further, the inventive composition prepared by using a separated granule of amlodipine or a pharmaceutically acceptable salt thereof provides improved dissolution rate and stability relative to the composition of a non-separated mixture of amlodipine and losartan when administered.

While the invention has been described with respect to the above specific embodiments, it should be recognized that various modifications and changes may be made to the invention by those skilled in the art which also fall within the scope of the invention as defined by the appended claims.

What is claimed is:

1. A composition for preventing or treating cardiovascular disorders comprising amlodipine or a pharmaceutically acceptable salt thereof and losartan or a pharmaceutically acceptable salt thereof.

2. The composition of claim 1, wherein amlodipine or a pharmaceutically acceptable salt thereof and losartan or a pharmaceutically acceptable salt thereof are physically separated from each other.

3. The composition of claim 2, which is prepared by a method comprising the steps of granulating amlodipine or a pharmaceutically acceptable salt thereof to obtain an amlodipine granule part, and mixing the amlodipine granule part with a mixture part comprising losartan or a pharmaceutically acceptable salt thereof.

4. The composition of claim 3, which further comprises a coating layer formed on the amlodipine granule part.

5. The composition of claim 3, which is in the form of a two-layer tablet in which amlodipine or a pharmaceutically acceptable salt thereof and losartan or a pharmaceutically acceptable salt thereof respectively form separate layers.

6. The composition of claim 3, wherein the amlodipine granule part and the mixture part further comprise pharmaceutically acceptable excipients.

7. The composition of claim 6, wherein the amlodipine granule part comprises amlodipine or a pharmaceutically acceptable salt thereof and pharmaceutically acceptable excipients in amounts corresponding to a weight ratio in the range of 1:10 to 1:60.

8. The composition of claim 2, which is prepared by a method comprising the step of coating a tablet of losartan or a pharmaceutically acceptable salt thereof with a layer of amlodipine or a pharmaceutically acceptable salt thereof.

9. The composition of claim 8, which further comprises a separating layer disposed between the tablet of losartan or a pharmaceutically acceptable salt thereof and the coating layer of amlodipine or a pharmaceutically acceptable salt thereof.

10. The composition of claim 1, wherein the pharmaceutically acceptable salt of amlodipine is amlodipine camsylate.

11. The composition of claim 1, wherein the pharmaceutically acceptable salt of losartan is losartan potassium.

12. The composition of claim 1, wherein the cardiovascular disorders are selected from the group consisting of angina pectoris, hypertension, artery vasospasm, deep vein, cardiac hypertrophy, cerebral infarct, congestive heart failure and myocardial infarction.

13. A method for preparing the composition of claim 3, which comprises the steps of:
a) wet-granulating and drying a mixture of amlodipine or a pharmaceutically acceptable salt thereof and pharmaceutically acceptable excipients to obtain an amlodipine granule part; and
b) mixing the amlodipine granule part obtained in step a) with a mixture part comprising losartan or a pharmaceutically acceptable salt thereof and pharmaceutically acceptable excipients.
